(19) United States
(12) Patent Application Publication
(10) Pub. No.: US 2019/0374930 A1
(43) Pub. Date: Dec. 12, 2019

(54) SOLID PHOSPHORIC ACID CATALYSTS

(71) Applicant: Clariant Corporation, Louisville, KY (US)

(72) Inventors: Wayne Turbeville, Crestwood, KY (US); Greg Korynta, Louisville, KY (US); Peter Hogue, Louisville, KY (US); Wenqin Shen, Louisville, KY (US); Marc Born, Louisville, KY (US); David Tolle, Louisville, KY (US); Michael Severance, Louisville, KY (US); Axel Düker, Maisach (DE)

(21) Appl. No.: 16/481,886

(22) PCT Filed: Mar. 9, 2018
(86) PCT No.: PCT/US2018/021678

§ 371 Date: Jul. 30, 2019

Related U.S. Application Data
(60) Provisional application No. 62/470,313, filed on Mar. 12, 2017.

(51) Int. Cl. B01J 27/182; B01J 27/16; B01J 37/00; B01J 35/10; C07C 2/18

(52) U.S. Cl. CPC B01J 27/182; B01J 27/16; C07C 2/18; B01J 35/1038; B01J 37/0009

(57) ABSTRACT

The present disclosure relates to solid phosphoric acid (SPA) catalyst compositions useful in the formation of hydrocarbons, such as the oligomerization of olefins, prepared from formable mixtures that comprise a phosphate source and a siliceous support material source in amounts, for example, such that the ratio of the phosphate source and the siliceous support material source is within the range of about 2.9:1 to about 3.4:1 calculated on a weight basis as H₃PO₄:SiO₂, and a dry particulate material.

### CROSS-REFERENCE TO RELATED APPLICATIONS

This application claims the benefit of priority of U.S. Provisional Patent Application No. 62/470,313, filed Mar. 12, 2017, which is hereby incorporated herein by reference in its entirety.

### BACKGROUND OF THE DISCLOSURE

### Field of the Disclosure

This disclosure relates generally to solid catalyst materials. More particularly, the present disclosure relates to solid phosphoric acid (SPA) catalysts useful in the conversion of hydrocarbons, such as the oligomerization of olefins, to methods for making such SPA catalysts, and to methods for converting hydrocarbons comprising contacting hydrocarbons with such catalysts.

### Technical Background

Solid phosphoric acid (SPA) catalysts are known for their usefulness in various hydrocarbon conversion processes, such as the alkylation of benzene and other aromatic hydrocarbons with olefins to produce alkyl aromatic products such as cumene and ethylbenzene, and the oligomerization or polymerization of olefins, for example, the oligomerization of light olefins to heavier olefins and parrafins (“polymer gasoline” or “polygas”). Conventional SPA catalysts are made by calcining mixtures of one or more phosphoric acids with one or more siliceous support material sources. This typically results in a complex mixture of phosphoric acids (e.g., orthophosphoric acid, pyrophosphoric acid, triphosphoric acid), silicon phosphates formed by reaction of phosphoric acids with the siliceous support material source, and, in some cases, siliceous support material. The operative catalyst is typically a layer of liquid phosphoric acids on solid silicon phosphates; silicon orthophosphate may act as a reservoir of orthophosphoric acid, which is a desirable catalytic material.

However, conventional SPA catalysts are not particularly robust, and can degrade over time (e.g., via deactivation, disintegration, etc.). Moreover, catalytic performance must, in many cases, be balanced with the physical properties of the catalyst material. For example, increased amounts of phosphoric acids improve catalytic performance, but provide a catalyst material that lacks the physical properties necessary for sustained use. Over time, a process using a conventional SPA catalyst can require increased operational temperatures, lower reactor space velocities to maintain acceptable conversion levels. In turn, higher temperatures result in undesirable by-products and increased rates of coking of the catalyst, and slower flow rates result in lower overall rates of production. Accordingly, the use of conventional SPA catalysts requires relatively frequent reactor shut-downs in order to replace the SPA catalyst, all resulting in a decrease in overall process efficiency.

Accordingly, there remains a need for a more robust SPA catalyst with improvements in one or more areas of activity, crush strength, crystallinity, acidity (surface and/or total), and porosity.

### SUMMARY OF THE DISCLOSURE

One aspect of the disclosure relates to a method for preparing a solid phosphoric acid catalyst composition, the method comprising
providing a formable mixture comprising a phosphate source present in the formable mixture in an amount within the range of about 50 wt. % to about 85 wt. %, calculated as H₃PO₄;
a siliceous support material source present in the formable mixture in an amount within the range of about 8 wt. % to about 35 wt. %, calculated as SiO₂, for example, such that the ratio of the phosphate source to the siliceous support material source is within the range of about 2.9:1 to about 4.5:1, calculated on a weight basis as H₃PO₄:SiO₂; and
a dry particulate material present in the formable mixture in an amount within the range of about 2 wt % to about 20 wt %, the dry particulate material comprising silica;
   one or more silicon phosphates; and/or
   a mixture comprising one or more phosphoric acids, one or more silicon phosphates, and, optionally, a siliceous support material;
   wherein the amount of silicon in the dry particulate material is at least about 15 wt. %, calculated as SiO₂ on a calcined basis;
forming the mixture; and
calcining the formed mixture.

Another aspect of the disclosure is a catalyst composition made by a method as described herein. The catalyst composition can, for example, consist essentially of:
one or more phosphoric acids;
one or more silicon phosphates; and
optionally, a siliceous support material.

Another aspect of the disclosure is a calcined solid phosphoric acid catalyst composition comprising, for example, consisting essentially of:
one or more phosphoric acids
one or more silicon phosphates;
optionally, one or more additional inorganic phosphates; and
optionally, a siliceous support material,
wherein the amount of phosphorus in the calcined solid phosphoric acid catalyst composition is within the range of about 74.5 wt % to about 76.5 wt % calculated as H₃PO₄ on a calcined basis.
   Such materials can advantageously be made using the processes described herein.

Another aspect of the disclosure is a method for converting hydrocarbons, the method comprising contacting a hydrocarbon feed with a catalyst composition as described herein. The hydrocarbon conversion can be, for example, an olefin oligomerization or aromatic hydrocarbon alkylation.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the materials and processes of the disclosure in more detail than is necessary for a fundamental understanding, the description taken with the drawings and/or examples making apparent to those skilled in the art how the several forms of the disclosure may be embodied in practice. Thus, before the disclosed processes and devices are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the materials and processes of the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the materials and processes of the disclosure and does not pose a limitation on the scope of the disclosure otherwise claimed. No language in the specification should be construed as indicating any non-claimed element essential to the practice of the materials and processes of the disclosure.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the materials and processes of the disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

As used herein, the term “consists essentially of” means that the material is at least 90% (e.g., at least 95%, at least 98% or even at least 99%) of the recited components, and does not include a component sufficient to change the catalyst activity or stability by more than 10%, more than 5%, or more than 2%.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variations on the particular embodiments described embodiments will become apparent to those of ordinary skill in the art upon reading the foregoing description. Moreover, any combination of the above-described elements in all possible variations thereof is encompassed by the disclosure unless otherwise indicated herein or otherwise clearly contradicted by context.

Furthermore, numerous references have been made to patents and printed publications throughout this specification. Each of the cited references and printed publications are individually incorporated herein by reference in their entirety.

In closing, it is to be understood that the embodiments disclosed herein are illustrative of the principles of the materials and processes of the disclosure. Other modifications that may be employed are within the scope of the disclosure. Thus, by way of example, but not of limitation, alternative configurations of the present invention may be utilized in accordance with the teachings herein. Accordingly, the present disclosure is not limited to that precisely as shown and described.

The disclosure relates to SPA catalyst compositions prepared from formable mixtures that comprise a phosphate source and a siliceous support material source in amounts such that the ratio of the phosphate source and the siliceous support material source is within the range of about 2.9:1 to about 4.5:1, calculated on a weight basis as H₃PO₄:SiO₂, and a dry particulate material selected from one or more of silica, silicon phosphates, and/or a mixture of silicon phosphates and phosphoric acids optionally including siliceous support material). The dry particulate material can be, advantageously, a rework material from a prior SPA catalyst synthesis, or an SPA catalyst fines material. This disclosure demonstrates such SPA catalysts to exhibit especially high activity and good stability relative to other SPA catalysts prepared from formable mixtures lacking the dry particulate material and/or having relative amounts of the phosphate source and the siliceous support material source different than those disclosed herein, such as commercially available SPA catalysts.

One aspect of the disclosure is a method for preparing a SPA catalyst composition. The method includes providing a formable mixture comprising (i) a phosphate source present in an amount within the range of about 55 wt. % to about 80 wt. % (calculated as H₃PO₄), (ii) a siliceous support material source present in an amount within the range of about 10 wt. % to about 30 wt. % (calculated as SiO₂), for example, such that the ratio of the phosphate source to the siliceous support material source is within the range of about 2.9:1 to about 4.5:1 (calculated on a weight basis as H₃PO₄:SiO₂), and (iii) a dry particulate material present in an amount within the range of about 2 wt. % to about 20 wt. %. In certain advantageous embodiments as otherwise described herein, the dry particulate material includes one or more one or more phosphoric acids, one or more silicon phosphates, and, optionally, a siliceous support material; such material can be provided as rework material from a prior catalyst synthesis, or as catalyst fines. In other embodiments as otherwise described herein, the dry particulate material is silica. In still other embodiments as otherwise described herein, the dry particulate material is a silicon phosphate. And in other embodiments as otherwise described herein the dry particulate material includes one or more of the above-described materials. The method includes forming (e.g., by extruding, tableting or pelletizing) the mixture and calcining the formed mixture.

The formable mixture includes a phosphate source. In some embodiments of the disclosure as otherwise described herein, the phosphate source is phosphoric acid, a compound that forms phosphoric acid by hydrolysis, or any mixture thereof. The phosphoric acid may be in any oligomeric and/or polymeric state, e.g., linear phosphoric acids including orthophosphoric acid, pyrophosphoric acid, tripolyphosphoric acid, tetrapolyphosphoric acid, etc. (i.e., the Hₙ₊₂PₙO₃ₙ₊₁ series), branched polyphosphoric acids, or metaphosphoric acids including trimetaphosphoric acid, tetrametaphosphoric acid, etc. In some embodiments of the disclosure as otherwise described herein, free phosphoric acidic sites comprising the catalyst precursor material (i.e., Bronsted sites) may be deprotonated. For example, orthophosphoric acid may be present as phosphoric acid (H₃PO₄) or as one of the conjugate bases dihydrogen phosphate (H₂PO₄₋), hydrogen phosphate (HPO₄²⁻), or phosphate (PO₄³⁻). In some embodiments of the disclosure as otherwise described herein, the catalyst precursor material includes orthophosphoric acid and, optionally, one or more of pyrophosphoric acid, tripolyphosphoric acid, and tetrapolyphosphoric acid.

In some embodiments of the disclosure as otherwise described herein, the phosphate source contains linear phosphoric acids, e.g., in combination with water. The person of ordinary skill in the art will appreciate that this mixture is characterized by the total phosphorus content, which is given as a percentage relative to pure orthophosphoric acid, H₃PO₄. As the other acids in the linear phosphoric acid series (i.e., Hₙ₊₂PₙO₃ₙ₊₁) have a higher phosphorus content by weight than orthophosphoric acid, it is not unusual to find phosphoric acids with a concentration greater than 100%. In some embodiments of the disclosure as otherwise described herein, the phosphate source is phosphoric acid with a concentration within the range of about 90% to about 130%, e.g., about 95% to about 125%, or about 100% to about 120%, or about 105% to about 115%, or the concentration is about 100%, or about 105%, or about 110%, or about 115%, or about 120%.

The formable mixture includes a phosphate source present in an amount in the range of 50 wt. % to about 85 wt. %, calculated as H₃PO₄ (i.e., based on the total phosphorus content). In some embodiments of the methods as described herein, the formable mixture includes a phosphate source present in an amount in the range of about 55 wt. % to about 85 wt. %, or about 60 wt. % to about 85%, or about 50 wt. % to about 80 wt. %, or about 50 wt. % to about 75 wt. %, or about 55 wt. % to about 80 wt. %, or about 60 wt. % to about 75 wt. %, or in an amount of about 60 wt. %, or about 65 wt. %, or about 70 wt. %, or about 75 wt. %, calculated as H₃PO_{4.}

The formable mixture also includes a siliceous support material source. In some embodiments, the siliceous support material may be any SiO₂-containing material, e.g., diatomaceous earth, infusorial earth, ciliate earth, fuller's earth, kaolin, celite, artificial porous silica, etc. In some embodiments of the disclosure as otherwise described herein, the siliceous support material source may be any mixture of two or more SiO₂-containing materials. In some embodiments of the disclosure as otherwise described herein, the siliceous support material source includes diatomaceous earth. As the person of ordinary skill in the art will appreciate, the terms “diatomite”, “D.E.,” “kieselgur,” “kieselguhr,” and “guhr” are equivalent to diatomaceous earth. In certain embodiments of the disclosure as otherwise described herein, the siliceous support material source is substantially SiO₂, e.g., at least 80 wt. %, at least 90 wt. %, at least 95 wt. %, or at least 99 wt. % SiO₂. For example, in some embodiments of the disclosure as otherwise described herein, the siliceous support material source is diatomaceous earth, celite, artificial porous silica, and/or diatomaceous earth. In some particular embodiments, the siliceous support material source is diatomaceous earth. Of course, the person of ordinary skill in the art will appreciate that these siliceous support material sources can be present in a calcined form (i.e., the calcined product of any such material).

The formable mixture includes a siliceous support material source present in amount within the range of about 8 wt. % to about 35 wt. %.%, calculated as SiO₂ (i.e., based on the total silicon content). In some embodiments, the formable material includes a siliceous support material source present in an amount in the range of about 13 wt. % to about 35 wt. %, or about 18 wt. % to about 35 wt. %, or about 8 wt. % to about 30 wt. %, or about 8 wt. % to about 25 wt., or about 13 wt. % to about 30 wt. %, or about 18 wt. % to about 25 wt. %, or in an amount of about 18 wt. %, or about 20 wt. %, or about 25 wt. %, calculated as SiO₂.

In certain embodiments as otherwise described herein, the phosphate source and the siliceous support material source are included in the formable mixture in amounts such that the ratio of the phosphate source to the siliceous support material source is within the range of about 2.9:1 to about 4.5:1, calculated on a weight basis as H₃PO₄:SiO₂ (i.e., based on the total phosphorus and silicon content of the phosphate source and the siliceous support material source, respectively). In some embodiments, the ratio of the phosphate source to the siliceous support material included in the formable mixture is within the range of about 2.95:1 to about 4.5:1, or about 3:1 to about 4.5:1, or about 3.05:1 to about 4.5:1, or about 3.2:1 to about 4.5:1, or about 3.5:1 to about 4.5:1, or about 3.9:1 to about 4.5:1, or about 3.95:1 to about 4.5:1, or about 4.0:1 to about 4.5:1, or about 4.05:1 to about 4.5:1, or about 4.1:1 to about 4.5:1, or about 4.15:1 to about 4.5:1, or about 4.2:1 to about 4.5:1, or about 4.25:1 to about 4.5:1, or about 3.85:1 to about 4.45:1, or about 3.85:1 to about 4.4:1, or about 3.85:1 to about 4.35:1, or about 2.9:1 to about 4.3:1, or about 2.95:1 to about 4.3:1, or about 3:1 to about 4.3:1, or about 3.05:1 to about 4.3:1, or about 3.2:1 to about 4.3:1, or about 3.5:1 to about 4.3:1, or about 3.85:1 to about 4.3:1, or about 3.85:1 to about 4.25:1, or about 2.9:1 to about 4.1:1, or about 2.95:1 to about 4.1:1, or about 3:1 to about 4.1:1, or about 3.05:1 to about 4.1:1, or about 3.2:1 to about 4.1:1, or about 3.5:1 to about 4.1:1, or about 3.85:1 to about 4.1:1, or about 2.9:1 to about 3.7:1, or about 2.95:1 to about 3.7:1, or about 3:1 to about 3.7:1, or about 3.05:1 to about 3.7:1, or about 3.2:1 to about 3.7:1, or about or about 3.5:1 to about 3.7:1, or about 2.9:1 to about 3.4:1, or about 2.95:1 to about 3.4:1, or about 3:1 to about 3.4:1, or about 3.05:1 to about 3.4:1, or about 3.2:1 to about 3.4:1, or about 3.9:1 to about 4.45:1, or about 3.95:1 to about 4.4:1, or about 4.0:1 to about 4.35:1, or the ratio is about 3.05:1, or about 3.1:1, or about 3.15:1, or about 3.2:1, or about 3.25:1, or about 3.3:1, or about 3.5:1, or about 3.7:1, or about 3.95:1, or about 4.0:1, or about 4.05:1, or about 4.1:1, or about 4.15:1, or about 4.2:1, or about 4.25:1, or about 4.3:1, or about 4.35:1, or about 4.4:1, or about 4.45:1, calculated on a weight basis as H₃PO₄:SiO₂.

In certain particular embodiments as otherwise described herein, diatomaceous earth is used as the siliceous support material source, and the ratio of the phosphate source to the diatomaceous earth is about 2.9:1 to about 3.4:1, for example, about 2.95:1 to about 3.4:1, or about 3:1 to about 3.4:1, or about 3.05:1 to about 3.4:1, or about 2.9:1 to about 3.35:1, or about 2.9:1 to about 3.3:1, or about 2.9:1 to about 3.25:1, or about 2.95:1 to about 3.35:1, or about 3:1 to about 3.3:1, or about 3.05:1 to about 3.25:1, or the ratio is about 3.05:1, or about 3.1:1, or about 3.15:1, or about 3.2:1, or about 3.25:1, calculated on a weight basis as H₃PO₄:diatomaceous earth.

As described above, the formable mixture also includes a dry particulate material. The dry particulate material may, for example, include silica and/or one or more silicon phosphates, or may be a mixture comprising one or more phosphoric acids (e.g., orthophosphoric acid, pyrophosphoric acid, triphosphoric acid), one or more silicon phosphates, and, optionally, a siliceous support material.

The amount of silicon in the dry particulate material is at least about 15 wt. %, calculated as SiO₂ on a calcined basis (i.e., based on the total silicon content). In some embodiments as otherwise described herein, the amount of silicon in the rework component is within the range of about 15 wt. % to about 95 wt. %, or about 15 wt. % to about 90 wt. %, or about 15 wt. % to about 85 wt. %, or about 15 wt. % to about 80 wt. %, or about 15 wt. % to about 75 wt. %, or about 15 wt. % to about 70 wt. %, or about 15 wt. % to about 65 wt %, or about 15 wt. % to about 60 wt. % or about 20 wt. % to about 60 wt. %, or about 25 wt. % to about 60 wt. %, or about 15 wt. % to about 55 wt. %, or about 20 wt. % to about 55 wt. %, or about 25 wt. % to about 55 wt. %, or about 15 wt. % to about 50 wt. %, or about 20 wt. % to about 50 wt. %, or about 25 wt. % to about 50 wt. %, or about 15 wt. % to about 45 wt. %, or about 20 wt. % to about 45 wt. %, or about 25 wt. % to about 45 wt. %, or about 15 wt. % to about 40 wt. %, or about 20 wt. % to about 40 wt. %, or about 25 wt. % to about 40 wt. %, calculated as SiO₂ on a calcined basis. The amount of silicon can also be calculated based on the identities and amounts of materials used in making the dry particulate material (e.g., when it is a rework material or a catalyst fines material).

In some embodiments as otherwise described herein, the dry particulate material includes one or more silicon phosphates, i.e., silicon orthophosphate and, optionally, one or more of silicon pyrophospahte, silicon tripolyphosphate, and silicon tetrapolyphosphate. In some embodiments, the rework component includes at least 50 wt. % silicon phosphates, e.g., at least 55 wt. %, or at least 60 wt. %, or at least 65 wt. %, or at least 70 wt. %, or at least 75 wt. %, or at least 80 wt. %, or at least 85 wt. %, or at least 90 wt. %, or at least 95 wt. %, or at least 97.5 wt. %, or at least 99 wt. %, or at least 99.5 wt. %, or at least 99 wt. % silicon phosphates.

In some embodiments as otherwise described herein, the dry particulate material includes a mixture comprising one or more phosphoric acids (e.g., orthophosphoric acid, pyrophosphoric acid, triphosphoric acid), one or more silicon phosphates, and, optionally, a siliceous support material. In some embodiments as otherwise described herein, the dry particulate material substantially comprises the mixture, i.e., the dry particulate material includes at least 95 wt. %, 97.5 wt. %, 99 wt. %, 99.5 wt. %, or 99.9 wt. % of the mixture. In other embodiments as otherwise described herein, the dry particulate material comprises the mixture and silica or silicon phosphates. For example, in certain embodiments as otherwise described herein, the dry particulate material comprises the mixture in an amount within the range of 60 wt. % to about 95 wt. % and silica in the range of about 5 wt. % to about 40 wt. %.

The person of ordinary skill in the art will appreciate that the mixture comprising one or more phosphoric acids, one or more silicon phosphates, and, optionally, a siliceous support material may be the dried or calcined product of a mixture comprising a phosphate source and a silicon source. In certain advantageous embodiments, such a dry particulate material can be rework material from an earlier SPA catalyst preparation.

As described above, the dry particulate material may include one or more phosphoric acids. In some aspects, the phosphoric acid may be in any oligomeric and/or polymeric state, e.g., linear phosphoric acids including orthophosphoric acid, pyrophosphoric acid, tripolyphosphoric acid, tetrapolyphosphoric acid, etc. (i.e., the Hₙ₊₂PₙO₃ₙ₊₁ series), branched polyphosphoric acids, or metaphosphoric acids including trimetaphosphoric acid, tetrametaphosphoric acid, etc. The person of ordinary skill in the art will appreciate that, typically, there will be a plurality of different phosphoric acids present in the component, e.g., a mixture of two or more of the phosphoric acids specifically named above or other phosphoric acids. In some embodiments as otherwise described herein, the dry particulate material includes orthophosphoric acid and, optionally, one or more of pyrophosphoric acid, tripolyphosphoric acid, and tetrapolyphosphoric acid.

As described above, the dry particulate material may include one or more silicon phosphates. For example, in some embodiments as otherwise described herein, there is a significant amount of silicon phosphate(s) (e.g., formed by the reaction during calcining of a phosphate source and a siliceous support material source). In some embodiments as otherwise described herein, such phosphates may be in any oligomeric and/or polymeric state, e.g., linear phosphates including orthophosphate, pyrophosphate, tripolyphosphate, tetrapolyphosphate, etc., branched polyphosphates, or metaphosphates. In some embodiments as otherwise described herein, the dry particulate material includes silicon orthophosphate and, optionally, one or more of silicon pyrophosphate, silicon tripolyphosphate, and silicon tetrapolyphosphate. The phosphates may be in any state of deprotonation; for example, orthophosphate may be dihydrogen phosphate (H₂PO₄₋), hydrogen phosphate (HPO₄²⁻), or phosphate (PO₄³⁻).

In some embodiments as otherwise described herein, the amount of phosphate in the dry particulate material is within the range of about 30 wt. % to about 85 wt. %, calculated as P₂O₅ on a calcined basis (i.e., based on the total phosphorus content). In some embodiments as otherwise described herein, the amount of phosphate in the dry particulate material is within the range of about 30 wt. % to about 75 wt. %, or about 40 wt. % to about 85 wt. %, or about 40 wt. % to about 80 wt. %, or about 40 wt. % to about 75 wt. %, or about 45 wt. % to about 85 wt. %, or about 45 wt. % to about 80 wt. %, or about 45 wt. % to about 75 wt. %, or about 50 wt. % to about 85 wt. %, or about 50 wt. % to about 80 wt. %, or about 50 wt. % to about 75 wt. %, or about 55 wt. % to about 85 wt. %, or about 55 wt. % to about 80 wt. %, or about 55 wt. % to about 75 wt. %, or about 60 wt. % to about 85 wt. %, or about 60 wt. % to about 80 wt. %, or about 60 wt. % to about 75 wt. %, calculated as P₂O₅ on a calcined basis. The person of ordinary skill in the art will quantify the amount of phosphoric acid and/or inorganic phosphate using conventional methodologies in the art, e.g., XRD, pH titration and ³¹P NMR. The amount of phosphate can also be calculated based on the identities and amounts of materials used in making the dry particulate material.

The person of ordinary skill in the art will appreciate that the dry particulate material can include a significant amount of silicon phosphates. As described above, the phosphate content will be quantified as P₂O₅ as described above, while the silicon content will be quantified as SiO₂ as described above.

In many embodiments as otherwise described herein, substantially no siliceous support material (i.e., other than the one or more silicon phosphates) is present in the dry particulate material. For example, in certain embodiments as otherwise described herein, there is less than 1 wt. %, less than 0.5 wt. % or less than 0.1 wt. % (calculated as SiO₂) siliceous support material (i.e., other than the silicon phosphates).

In certain desirable embodiments, the dry particulate material includes phosphorus in the range of 70 wt. % to 80 wt. %, calculated as H₃PO₄, and silicon in the range of 20-30 wt. %, calculated as SiO₂, both on a calcined basis. For example, in certain embodiments, the dry particulate material includes phosphorus in the range of 72.5 wt. % to 78 wt. %, calculated as H₃PO₄, and silicon in the range of 22 wt. % to 27.5 wt. %, calculated as SiO₂, both on a calcined basis.

The dry particulate material is “dry”; while it may be calcined, however, it need not be so. In many embodiments as otherwise described herein, substantially no water is present in the dry particulate material. For example, in certain desirable embodiments as otherwise described herein, the dry particulate material is a calcined material. In some embodiments, there is less than 5 wt. %, less than 2 wt. %, less than 1 wt. %, less than 0.5 wt. %, or less than 0.1 wt. % water present in the dry particulate material.

In some embodiments as otherwise described herein, the free acidity of the dry particulate material is within the range of about 10% to about 40%, e.g., about 10% to about 35%, or about 10% to about 30%, or about 10% to about 25%, or about 15% to about 40%, or about 15% to about 35%, or about 15% to about 30%, or about 15% to about 25%, or about 20% to about 40%, or about 20% to about 35%, or about 20% to about 30%, or about 20% to about 25%, calculated as P₂O₅. Free acidity can be determined by the person of ordinary skill in the art, for example, using pH titration.

In some embodiments as otherwise described herein, the atomic molar ratio of phosphorus to silicon in the dry particulate material is within the range of about 0.25:1 to about 6:1, e.g., about 0.5:1 to about 6:1, or about 1:1 to 6:1, or about 2:1 to about 6:1, or about 3:1 to about 6:1, or about 4:1 to about 6:1, or about 0.25:1 to about 5:1, or about 0.5:1 to about 5:1, or about 1:1 to 5:1, or about 2:1 to about 5:1, or about 3:1 to about 5:1, or about 4:1 to about 5:1, or about 0.25:1 to about 4:1, or about 0.5:1 to about 4:1, or about 1:1 to 4:1, or about 2:1 to about 4:1, or about 3:1 to about 4:1, or about 0.25:1 to about 3:1, or about 0.5:1 to about 3:1, or about 1:1 to 3:1, or about 2:1 to about 3:1, or about 0.25:1 to about 2:1, or about 0.5:1 to about 2:1, or about 1:1 to 2:1. The amount of phosphorus and silicon can also be calculated based on the identities and amounts of materials used in making the dry particulate material.

The person of ordinary skill in the art will appreciate that the dry particulate material of the formable mixture may, in some especially desirable embodiments as otherwise described herein, be “rework material” or “catalyst fines,” that is, catalyst products, scrap pieces, fines, and/or rejected materials obtained from the process of making a calcined SPA catalyst composition such as that described in U.S. Pat. Nos. 7,557,060; 9,403,149; or even the dried intermediate or calcined product of the methods described herein.

In some embodiments as otherwise described herein, at least 70 wt. % of the dry particulate material is particles having a diameter of less than about 1 mm, e.g., less than about 0.95 mm, or less than about 0.9 mm, or less than about 0.85 mm, or less than about 0.8 mm, or less than about 0.75 mm, or less than about 0.7 mm, or less than about 0.65 mm, or less than about 0.6 mm, or less than about 0.55 mm, or less than about 0.5 mm, or less than about 0.45 mm. For example, in certain embodiments as otherwise described herein, at least 20 wt. % of the dry particulate material comprises particles having diameter of less than about 0.11 mm and at least 40 wt. % of the dry particulate material comprising particles having a diameter between about 0.11 and 0.85.

The formable mixture includes the dry particulate material in an amount within the range of about 2 wt. % to about 20 wt. %. In some embodiments as otherwise described herein, the formable mixture includes a dry particulate material in an amount within the range of about 2 wt. % to about 19 wt. %, or about 2 wt. % to about 18 wt. %, or about 2 wt. % to about 17 wt. %, or about 2 wt. % to about 16 wt. %, or about 2 wt. % to about 15 wt. %, or about 3 wt. % to about 20 wt. %, or about 4 wt. % to about 20 wt. %, or about 5 wt. % to about 20 wt. %, or in an amount of about 5 wt. %, or about 6 wt. %, or about 7 wt. %, or about 8 wt. %, or about 9 wt. %, or about 10 wt. %, or about 11 wt. %, or about 12 wt. %, or about 13 wt. %, or about 14 wt. %, or about 15 wt. %.

The person of ordinary skill in the art will appreciate that, in some embodiments as otherwise described herein, other conventional materials can be included in the formable mixture, e.g., water, binders, cements, or any of other materials to aid with mixing or forming (e.g., via extrusion, pelleting or tabletting). But in other embodiments, the calcinable solids of the formable mixture consist essentially of the phosphate source, the siliceous support material source, and the embodiments as otherwise described herein (i.e., provided along with any water necessary to make the mixture formable). For example, in certain such embodiments, the calcinable solids of the formable mixture are at least 95%, at least 98%, at least 99%, or at least 99.5% by calcined weight of the phosphate source, the siliceous support material source, and the dry particulate material.

In some embodiments as otherwise described herein, the total amount of phosphorus, silicon, oxygen, and hydrogen is at least about 95 wt. % of the formable mixture on a calcined weight basis, e.g., at least about 96 wt. %, or at least about 97 wt. %, or at least about 97.5 wt. % or at least about 98 wt. %, or at least about 98.5 wt. %, or at least about 99 wt. %, or at least about 99.5 wt. %, or at least about 99.9 wt. % of the formable mixture on a calcined weight basis. Notably, the presently-disclosed materials and processes can provide superior SPA catalyst performance without the use of promoter elements.

As the person of ordinary skill in the art will appreciate, diatomaceous earth can include small amounts of aluminum and iron. In some embodiments as otherwise described herein, the total amount of phosphorus, silicon, oxygen, aluminum, iron and hydrogen is at least about 95 wt. % of the formable mixture on a calcined weight basis, e.g., at least about 96 wt. %, or at least about 97 wt. %, or at least about 97.5 wt. % or at least about 98 wt. %, or at least about 98.5 wt. %, or at least about 99 wt. %, or at least about 99.5 wt. %, or at least about 99.9 wt. % of the formable mixture on a calcined weight basis, in which the amount of iron is no more than about 1 wt. %, no more than about 0.5 wt %, or no more than about 0.25 wt %, on a calcined weight basis, and the amount of aluminum is no more than about 2 wt. %, no more than about 1 wt %, or no more than about 0.5 wt %, on a calcined weight basis. Notably, the presently-disclosed materials and processes can provide superior SPA catalyst performance without the use of promoter elements.

In certain embodiments of the processes as otherwise described herein, the calcinable components of the formable mixture comprise at least 90% of (e.g., at least 95% of, at least 98% of or at least 99% of) a mixture of
65-85% by weight phosphoric acid having a concentration within the range of about 90% to about 130%; and
15-35% by weight diatomaceous earth.

In certain embodiments of the processes as otherwise described herein, the calcinable components of the formable mixture comprise at least 90% of (e.g., at least 95% of, at least 98% of or at least 99% of) a mixture of
70-80% by weight phosphoric acid having a concentration within the range of about 90% to about 130%; and
20-30% by weight diatomaceous earth.

The “calcinable components” are those that leave behind a substantial inorganic residue upon calcination. Accordingly, they include the phosphate source and the siliceous support material source, as well as any metal-containing components, but do not include water, other solvents, or pore-forming agents.

The person of ordinary skill in the art will appreciate that the amounts of material in the calcined formed material are to be calculated on an as-calcined basis, exclusive of any organic material and any adsorbed water.

The person of ordinary skill in the art will further appreciate that the forms of the phosphate source, siliceous support material source, and dry particulate material in the formable material may be varied and combined in a number of ways.

The person of the ordinary skill in the art will also appreciate that the order of addition of the phosphate source, siliceous support material source, and dry particulate material may vary in a number of ways. In one example, the phosphate source and siliceous support material source are mixed together before the dry particulate material is added. In another example, the siliceous support material source and the dry particulate material are mixed together before the phosphate source is added. In another example, the phosphate source and the dry particulate material are mixed together before the siliceous support material source is added.

The components of the formable mixture may be mixed by a variety of methods, both manual and mechanical. In some embodiments as otherwise described herein, two or more components of the formable mixture are mixed by hand. In some embodiments as otherwise described herein, two or more components of the formable mixture are mixed mechanically. In some embodiments as otherwise described herein, the mechanical mixing may be accomplished using, e.g., a planetary mixer, a spiral mixer, a stand mixer, screw extruder etc. In some embodiments as otherwise described herein, the formable mixture may be mixed by a combination of hand and mechanical mixing.

The method of preparing an SPA catalyst composition may optionally include a precalcining step before the formable mixture is formed. As used herein, the term “precalcine” describes the first heating step in a process in which there are at least two heating steps (i.e., a material may be precalcined, then calcined). In some aspects, the precalcination step may be performed at a temperature lower than that of the calcination step. Precalcining can be performed, e.g., to dry the bulk of the water out of the formable mixture in advance of the calcining step. In some embodiments as otherwise described herein, the formable mixture comprising the phosphate source, siliceous support material source, and dry particulate material is precalcined before it is formed. In some embodiments as otherwise described herein, the formable mixture is precalcined at a temperature within the range of about 50° C. to about 350° C., e.g., about 75° C. to about 325° C., or about 100° C. to about 300° C., or about 125° C. to about 275° C., or about 150° C. to about 250° C., or about 175° C. to about 225° C., or the temperature is about 100° C., or about 125° C., or about 150° C., or about 175° C., or about 200° C., or about 225° C., or about 250° C., or about 275° C., or about 300° C. After such a drying step, the material may be suitable for use as a rework material in a later catalyst manufacture process.

In some embodiments as otherwise described herein, the formable mixture is precalcined for a period of time within the range of 5 min. to about 2 hr., e.g., about 5 min. to about 1.5 hr., or about 5 min. to about 1 hr., or about 5 min. to about 50 min., or about 5 min. to about 35 min., or about 10 min. to about 30 min., or about 15 min. to about 25 min., or the period of time is about 5 min., or about 10 min., or about 15 min., or about 20 min., or about 25 min., or about 30 min., or about 35 min., or about 40 min., or about 45 min.

After a precalcining step, it will often be desirable to rehydrate the mixture in order to ensure it is formable for the forming step. Organic binders and extrusion aids can be advantageously added after precalcining.

It can be advantageous to add a material which produces gases during calcination, as this aids in the formation of the large pores which characterize this catalyst. Materials which produce gases during calcination include, without limitation, materials such as water or other volatiles which produce gas by evaporation or loss on ignition, and organic or inorganic materials such as those containing starch, cellulose, nitrates, carbonates, oxalates, acetates or other organic salts, polymers, or compounds containing coordinated water or ammonia, which produce gas by decomposition or combustion. In certain embodiments, a pore-forming organic material (e.g., polyethylene glycol, maize flour) is added to the precalcined mixture before forming the SPA catalyst composition. The pore-forming organic material can be burned away during the calcining step, leaving pores behind. The use of pore-forming organic materials is familiar to the person of ordinary skill in the art.

The method of preparing an SPA catalyst composition includes forming the optionally-precalcined formable mixture. The person of ordinary skill in the art will appreciate that the optionally precalcined formable mixture may be formed into a variety of shapes, e.g., extrudates, pellets, tablets, spheres, powder, etc. A variety of methods for forming such shapes are known in the art, e.g., extrusion, pelletizing, marumarizing, spray drying, etc. In certain particular embodiments as otherwise described herein, the formable mixture is formed by extrusion into an extrudate.

The method of preparing an SPA catalyst composition also includes calcining the formed mixture. In some embodiments as otherwise described herein, the calcination step may be performed at a temperature higher than that of the precalcination step. In some embodiments as otherwise described herein, the formed catalyst precursor material is calcined at a temperature within the range of about 120° C. to about 520° C., e.g., about 150° C. to about 490° C., or about 180° C. to about 460° C., or about 210° C. to about 430° C., or about 240° C. to about 400° C., or about 260° C. to about 380° C., or about 280° C. to about 360° C., or about 300° C. to about 340° C., or the temperature is about 240° C., or about 250° C., or about 260° C., or about 270° C., or about 280° C., or about 290° C., or about 300° C., or about 310° C., or about 320° C., or about 330° C., or about 340° C., or about 350° C., or about 360° C., or about 380° C., or about 400° C.

In some embodiments as otherwise described herein, the formed mixture is calcined for a period of time within the range of 5 min. to about 2.5 hr., e.g., about 5 min. to about 2 hr., or about 5 min. to about 1.5 hr., or about 5 min. to about 1 hr., or about 5 min. to about 55 min., or about 10 min. to about 50 min., or about 15 min. to about 45 min., or about 20 min. to about 40 min., or about 25 min. to about 35 min., or the period of time is about 10 min., or about 15 min., or about 20 min., or about 25 min., or about 30 min., or about 35 min., or about 40 min., or about 45 min., or about 50 min.

The person of ordinary skill in the art will select calcination conditions, including, possibly, multiple calcination steps at different times, temperatures, oxygen levels and moisture levels, to provide the desired material. The formed mixture may be calcined in two or more stages, with each stage having its own time, temperature, oxygen level, and moisture level. For example, the formed mixture may be dried at 120° C. for 1 hour in dry air, calcined at 400° C. for 1.5 hours in dry air, and then steamed at 200° C. for 0.5 hours in a 4:1 mixture of air and steam. However, it is not necessary to employ multiple calcination stages: a single stage in which the formed mixture held at a constant temperature for a certain amount of time may also be used.

The initial, “green” formed mixture is typically amorphous, and must undergo crystallization to produce the finished catalyst. Crystallization can occur in the period between mixing the ingredients and forming, in the period between forming and calcination, and/or during calcination.

The calcination temperature and calcination time should be sufficient to ensure growth of the crystalline phases of silicon orthophosphate and silicon pyrophosphate and the desired pore characteristics. Calcination temperatures above 500° C. contribute to excessive formation of silicon pyrophosphate and insufficient formation of silicon orthophosphate. In order to obtain a mixture of silicon orthophosphate and silicon pyrophosphate, the calcination temperature (or highest calcination temperature, if there are multiple calcination stages) should be in the range between about 200° C. and about 500° C., preferably between about 350° C. and about 450° C. Calcination times (total times, if there is more than one calcination stage) will vary depending on other calcination factors, but calcination times between about 20 minutes and about 4 hours are preferred.

In some embodiments as otherwise described herein, the method of preparing an SPA catalyst composition also includes a step of surface coating the calcined SPA catalyst composition. In some aspects, the calcined SPA catalyst may be surface coated with any SiO₂-containing material, e.g., diatomaceous earth, infusorial earth, ciliate earth, fuller's earth, kaolin, celite, artificial porous silica, etc. In some embodiments as otherwise described herein, the calcined SPA catalyst composition is surface coated with diatomaceous earth.

Another aspect of the disclosure is a SPA catalyst composition made by any method as described herein.

Another aspect of the disclosure is a calcined solid phosphoric acid catalyst composition comprising (e.g., consisting essentially of):
one or more phosphoric acids
one or more silicon phosphates;
optionally, one or more additional inorganic phosphates; and
optionally, a siliceous support material,
wherein the amount of phosphorus in the calcined solid phosphoric acid catalyst composition is within the range of about 74.5 wt % to about 76.5 wt % calculated as H₃PO₄ on a calcined basis.
   Such materials can advantageously be made using the processes described herein. The amount of phosphorus can be, for example about 75.0 wt. % to about 76.5 wt %, or about 75.0 wt. % to about 76.0 wt. %, or about 74.5 wt. % to about 76.0 wt. %. The present inventors have determined that the use of a dry particulate material in the synthesis of catalysts can allow for the stable synthesis of SPA catalysts with such high amounts of phosphorus.

SPA catalyst compositions of the disclosure include one or more phosphoric acids, one or more silicon phosphates, and optionally, a siliceous support material. In some aspects, the phosphoric acid may be in any oligomeric and/or polymeric state, e.g., linear phosphoric acids including orthophosphoric acid, pyrophosphoric acid, tripolyphosphoric acid, tetrapolyphosphoric acid, etc. (i.e., the Hₙ₊₂PₙO₃ₙ₊₁ series), branched polyphosphoric acids, or metaphosphoric acids including trimetaphosphoric acid, tetrametaphosphoric acid, etc. The person of ordinary skill in the art will appreciate that in typical catalyst samples there will be a plurality of different phosphoric acids present, e.g., a mixture of two or more of the phosphoric acids specifically named above or other phosphoric acids. In some embodiments, the SPA catalyst composition includes orthophosphoric acid and, optionally, one or more of pyrophosphoric acid, tripolyphosphoric acid, and tetrapolyphosphoric acid.

As described above, the compositions include one or more silicon phosphates. For example, in typical samples there is a significant amount of silicon phosphate(s), formed by the reaction during calcining of a phosphate source and a siliceous support material source. In some aspects, such phosphates may be in any oligomeric and/or polymeric state, e.g., linear phosphates including orthophosphate, pyrophosphate, tripolyphosphate, tetrapolyphosphate, etc., branched polyphosphates, or metaphosphates. In some embodiments, the SPA catalyst composition includes silicon orthophosphate and, optionally, one or more of silicon pyrophosphate, silicon tripolyphosphate, and silicon tetrapolyphosphate. The phosphates may be in any state of deprotonation; for example, orthophosphate may be dihydrogen phosphate (H₂PO₄₋), hydrogen phosphate (HPO₄²⁻), or phosphate (PO₄³⁻).

The person of ordinary skill in the art will appreciate that the ratio of silicon orthophosphate to silicon pyrophosphate may be determined from an integrated X-ray diffraction (XRD) reflectance ratio. Such a ratio is a comparison of the X-ray reflection intensities generated by the (113) planes of silicon orthophosphate and the (002) planes of silicon pyrophosphate. In some embodiments as otherwise described herein, the XRD reflectance intensity ratio of silicon orthophosphate to silicon pyrophosphate of the SPA catalyst composition is at least about 1.5:1, e.g., at least about 2:1, at least about 3:1, at least about 4:1, at least about 5:1, at least about 6:1, at least about 7:1, or at least about 8:1.

In some embodiments as otherwise described herein, the amount of phosphate in the SPA catalyst composition is within the range of about 30 wt. % to about 85 wt. %, calculated as P₂O₅ on a calcined basis. In some embodiments of the compositions as described herein, the amount of phosphate in the SPA catalyst composition is in the range of about 30 wt. % to about 80 wt. %, or about 30 wt. % to about 75 wt. %, or about 40 wt. % to about 85 wt. %, or about 40 wt. % to about 80 wt. %, or about 40 wt. % to about 75 wt. %, or about 45 wt. % to about 85 wt. %, or about 45 wt. % to about 80 wt. %, or about 45 wt. % to about 75 wt. %, or about 50 wt. % to about 85 wt. %, or about 50 wt. % to about 80 wt. %, or about 50 wt. % to about 75 wt. %, or about 55 wt. % to about 85 wt. %, or about 55 wt. % to about 80 wt. %, or about 55 wt. % to about 75 wt. %, or about 60 wt. % to about 85 wt. %, or about 60 wt. % to about 80 wt. %, or about 60 wt. % to about 75 wt. %, calculated as P₂O₅ on a calcined basis. Of course, in materials having phosphorus in within the range of about 74.5 wt % to about 76.5 wt % calculated as H₃PO₄ on a calcined basis, the full range of the above-referenced amounts of phosphate may not be available. The person of ordinary skill in the art will quantify the amount of phosphoric acid and/or inorganic phosphate using conventional methodologies in the art, e.g., XRD, pH titration and ³¹ P NMR. The amount of phosphate can also be calculated based on the identities and amounts of materials used in making the SPA catalyst composition.

In some embodiments as otherwise described herein, the free acidity of the SPA catalyst composition is within the range of about 10% to about 40%, e.g., about 10% to about 35%, or about 10% to about 30%, or about 10% to about 25%, or about 15% to about 40%, or about 15% to about 35%, or about 15% to about 30%, or about 15% to about 25%, or about 20% to about 40%, or about 20% to about 35%, or about 20% to about 30%, or about 20% to about 25%, calculated as P₂O₅. Free acidity can be determined by the person of ordinary skill in the art, for example, using pH titration.

In many embodiments as otherwise described herein, substantially no siliceous support material (i.e., other than the one or more silicon phosphates) is present in the SPA catalyst composition. As the person of ordinary skill in the art will appreciate, in many cases the siliceous support material source in the formable mixture is converted substantially completely to silicon phosphate when the formed mixture is calcined. For example, in certain embodiments as otherwise described herein, the SPA catalyst composition comprises less than 1 wt. %, less than 0.5 wt. % or less than 0.1 wt. % (calculated as SiO₂) siliceous support material (i.e., other than the one or more silicon phosphates).

However, as described above, the SPA catalyst composition can also optionally include a siliceous support material (i.e., in addition to the silicon present as silicon phosphate).

In certain embodiments as otherwise described herein, the siliceous support material is substantially SiO₂, e.g., at least 80 wt. %, at least 90 wt. %, at least 95 wt. %, or at least 99 wt. % SiO₂. For example, in some embodiments as otherwise described herein, the siliceous support material includes diatomaceous earth, celite, or artificial porous silica. In some particular embodiments as otherwise described herein the siliceous support material includes diatomaceous earth. Of course, the person of ordinary skill in the art will appreciate that these siliceous support materials can be present in a calcined form (i.e., as the calcined product of any such material).

In certain embodiments of the compositions as otherwise described herein, the amount of silicon in the SPA catalyst composition is within the range of about 15 wt. % to about 85 wt. % calculated as SiO₂ on a calcined basis. In some embodiments as otherwise described herein, the amount of silicon in the SPA catalyst composition is in the range of about 20 wt. % to about 70 wt. %, about 25 wt. % to about 70 wt. %, or about 15 wt. % to about 60 wt. %, or about 20 wt. % to about 60 wt. %, or about 25 wt. % to about 60 wt. %, or about 15 wt. % to about 55 wt. %, or about 20 wt. % to about 55 wt. %, or about 25 wt. % to about 55 wt. %, or about 15 wt. % to about 50 wt. %, or about 20 wt. % to about 50 wt. %, or about 25 wt. % to about 50 wt. %, or about 15 wt. % to about 45 wt. %, or about 20 wt. % to about 45 wt. %, or about 25 wt. % to about 45 wt. %, or about 15 wt. % to about 40 wt. %, or about 20 wt. % to about 40 wt. %, or about 25 wt. % to about 40 wt. %, calculated as SiO₂ on a calcined basis. Of course, in materials having phosphorus in within the range of about 74.5 wt % to about 76.5 wt % calculated as H₃PO₄ on a calcined basis, the full range of the above-referenced amounts of silicon may not be available.

The person of ordinary skill in the art will appreciate that the SPA catalyst composition can include a significant amount of silicon phosphates. As described above, the phosphate content will be quantified as P₂O₅ as described above, while the silicon content will be quantified as SiO₂ as described above.

In some embodiments as otherwise described herein, the atomic molar ratio of phosphorus to silicon in the SPA catalyst composition is within the range of about 0.25:1 to about 6:1, e.g., about 0.5:1 to about 6:1, or about 1:1 to 6:1, or about 2:1 to about 6:1, or about 3:1 to about 6:1, or about 4:1 to about 6:1, or about 0.25:1 to about 5:1, or about 0.5:1 to about 5:1, or about 1:1 to 5:1, or about 2:1 to about 5:1, or about 3:1 to about 5:1, or about 4:1 to about 5:1, or about 0.25:1 to about 4:1, or about 0.5:1 to about 4:1, or about 1:1 to 4:1, or about 2:1 to about 4:1, or about 3:1 to about 4:1, or about 0.25:1 to about 3:1, or about 0.5:1 to about 3:1, or about 1:1 to 3:1, or about 2:1 to about 3:1, or about 0.25:1 to about 2:1, or about 0.5:1 to about 2:1, or about 1:1 to 2:1. Of course, in materials having phosphorus in within the range of about 74.5 wt % to about 76.5 wt % calculated as H₃PO₄ on a calcined basis, the full range of the above-referenced P:Si ratios may not be available.

In some embodiments, the total amount of phosphorus, silicon, oxygen, and hydrogen is at least about 95 wt. % of the SPA catalyst composition on a calcined weight basis, e.g., at least about 96 wt. %, or at least about 97 wt. %, or at least about 97.5 wt. % or at least about 98 wt. %, or at least about 98.5 wt. %, or at least about 99 wt. %, or at least about 99.5 wt. %, or at least about 99.9 wt. % of the SPA catalyst composition on a calcined weight basis. Notably, the presently-disclosed materials and processes can provide superior SPA catalyst performance without the use of promoter elements.

As the person of ordinary skill in the art will appreciate, diatomaceous earth can include small amounts of aluminum and iron. In some embodiments as otherwise described herein, the total amount of phosphorus, silicon, oxygen, aluminum, iron and hydrogen is at least about 95 wt. % of the SPA catalyst composition on a calcined weight basis, e.g., at least about 96 wt. %, or at least about 97 wt. %, or at least about 97.5 wt. % or at least about 98 wt. %, or at least about 98.5 wt. %, or at least about 99 wt. %, or at least about 99.5 wt. %, or at least about 99.9 wt. % of the SPA catalyst composition on a calcined weight basis, in which the amount of iron is no more than about 1 wt. %, no more than about 0.5 wt %, or no more than about 0.25 wt %, on a calcined weight basis, and the amount of aluminum is no more than about 2 wt. %, no more than about 1 wt %, or no more than about 0.5 wt %, on a calcined weight basis. Notably, the presently-disclosed materials can provide superior SPA catalyst performance without the use of promoter elements.

The SPA catalyst composition produced by the methods described herein comprises pores, and is characterized both by the total pore volume and distribution of pore diameters. In some embodiments as otherwise described herein, the total pore volume of the SPA catalyst composition is at least 0.17 cm³, e.g., at least 0.18 cm³, or at least 0.19 cm³, or at least 0.20 cm³. In some embodiments, the volume contributed by pores having a diameter of at least 1 μm, e.g., at least 2.5 μm, at least 5 μm, or at least 10 μm, is at least 0.15 cm³. The person of ordinary skill in the art will appreciate that pore volume may be determined from mercury porosimetry.

Another embodiment of the disclosure is a method of converting hydrocarbons. The method includes providing a SPA catalyst composition as described herein. The method also includes contacting a hydrocarbon feed with the provided SPA catalyst composition. In some aspects, the hydrocarbon conversion may be oligomerization of an olefin, e.g., propylene oligomerization, butene oligomerization, etc. In some aspects, the hydrocarbon conversion may be alkylation of an aromatic hydrocarbon, e.g., benzene alkylation, etc. In some embodiments, the hydrocarbon conversion is olefin oligomerization.

The SPA catalyst compositions of the present disclosure may be used, for example, in the alkylation of aromatic hydrocarbons with olefins to produce alkyl aromatics. In one embodiment as otherwise described herein, benzene is reacted with ethylene to produce ethylbenzene. In another embodiment as otherwise described herein, benzene is reacted with propylene to produce cumene. In a typical process, the aromatic hydrocarbon and the olefin are continuously fed into a pressure vessel containing the solid phosphoric acid catalyst of this disclosure. The feed admixture may be introduced into the alkylation reaction zone containing the alkylation catalyst at a constant rate, or alternatively, at a variable rate. Normally, the aromatic substrate and olefinic alkylating agent are contacted at a molar ratio of from about 1:1 to 20:1 and preferably-from about 2:1 to 8:1. The preferred molar feed ratios help to maximize the catalyst life cycle by minimizing the deactivation of the catalyst by coke and heavy material deposition upon the catalyst. The catalyst may be contained in one bed within a reactor vessel or divided up among a plurality of beds within a reactor. The alkylation reaction system may contain one or more reaction vessels in series. The feed to the reaction zone can flow vertically upwards, or downwards through the catalyst bed in a typical plug flow reactor, or horizontally across the catalyst bed in a radial flow type reactor. A controlled amount of water, in quantities between about 0.01% and about 6% of the combined aromatic and olefin feed, is preferably added to the alkylation reaction zone, in order to prevent dehydration of the catalyst, which affects catalyst performance.

The SPA catalyst compositions of the present disclosure may also be used in a polygas process. In this process, sometimes called catalytic condensation, olefins in the feed stream are oligomerized to produce heavier hydrocarbons. In an exemplary embodiment, the particles of the catalyst are placed in vertical cylindrical treating towers or in fixed beds in reactors or towers and the gases containing olefins are passed downwardly through the reactors or towers at temperatures of 170° C. to 290° C. and pressures of 6 to 102 atmospheres. These conditions are particularly applicable when dealing with olefin-containing material which may contain from approximately 10 to 50 percent or more of propylene and butylenes. When operating on a mixture comprising essentially propylene and butylenes, preferred process conditions are a temperature from about 140° C. to about 250° C., and at a pressure of from about 34 to about 102 atmospheres.

In some aspects, the hydrocarbon feed may include any C3 or C4 hydrocarbon. In some aspects, the hydrocarbon may include saturated or unsaturated (i.e., olefinic) hydrocarbons. As the person of ordinary skill in the art will appreciate, the hydrocarbon feed may include a number of combinations of C3 and C4 hydrocarbons, and a number of combinations of saturated and olefinic hydrocarbons. In some embodiments, the hydrocarbon feed includes propylene. In some embodiments, the hydrocarbon feed includes 1-butene.

In some embodiments, the hydrocarbon feed includes an olefinic hydrocarbon present in an amount within the range of about 5 wt. % to about 95 wt. %, e.g., about 10 wt. % to about 90 wt. %, or about 15 wt. % to about 85 wt. %, or about 20 wt. % to about 80 wt. %, or about 20 wt. % to about 70 wt. %, or about 20 wt. % to about 60 wt. %, or about 20 wt. % to about 50 wt. %, or about 20 wt. % to about 40 wt. %, or about 30 wt. % to about 80 wt. %, or about 35 wt. % to about 75 wt. %, or about 40 wt. % to about 70 wt. %, or about 45 wt. % to about 65 wt. %, or the amount is about 15 wt. %, or about 20 wt. %, or about 25 wt. %, or about 30 wt. %, or about 35 wt. %, or about 40 wt. %, or about 45 wt. %, or about 50 wt. %, or about 55 wt. %, or about 60 wt. %, or about 65 wt. %, or about 70 wt. %.

In some embodiments, the hydration level of the hydrocarbon feed is within the range of about 50 ppm to about 1000 ppm, e.g., about 100 ppm to about 900 ppm, or about 150 ppm to about 850 ppm, or about 200 ppm to about 800 ppm, or about 250 ppm to about 750 ppm, or about 300 ppm to about 700 ppm, or about 350 ppm to about 650 ppm, or about 400 ppm to about 600 ppm, or about 450 ppm to about 550 ppm, or the hydration level is about 200 ppm, or about 250 ppm, or about 300 ppm, or about 350 ppm, or about 400 ppm, or about 450 ppm, or about 500 ppm, or about 550 ppm, or about 600 ppm, or about 650 ppm, or about 700 ppm.

In some embodiments, the hydrocarbon is contacted with the provided SPA catalyst composition at a liquid hourly space velocity of about 0.1 h⁻¹ to about 5 h⁻¹, e.g., about 0.25 h⁻¹ to about 4.5 h⁻¹, or about 0.5 h⁻¹ to about 4 h⁻¹, or about 0.75 h⁻¹ to about 3.5 h⁻¹, or about 1 h⁻¹ to about 3 h⁻¹, or about 1 h⁻¹ to about 2.5 h⁻¹, or about 1 h⁻¹ to about 2 h⁻¹, or about 1 h⁻¹ to about 1.75 h⁻¹, or about 1 h⁻¹ to about 1.5 h⁻¹, or the liquid hourly space velocity is about 0.25 h⁻¹, or about 0.5 h⁻¹, or about 0.75 h⁻¹, or about 1 h⁻¹, or about 1.25 h⁻¹, or about 1.5 h⁻¹, or about 1.75 h⁻¹, or about 2 h⁻¹, or about 2.5 h⁻¹, or about 3 h⁻¹, or about 3.5 h⁻¹, or about 4 h⁻¹.

In some embodiments, the method of converting hydrocarbons is carried out at a temperature within the range of about 50° C. to about 450° C., e.g., about 75° C. to about 400° C., or about 100° C. to about 350° C., or about 100° C. to about 300° C., or about 100° C. to about 250° C., or about 100° C. to about 200° C., or about 125° C. to about 175° C., or the temperature is about 100° C., or about 120° C., or about 140° C., or about 160° C., or about 180° C., or about 200° C., or about 220° C., or about 240° C., or about 260° C., or about 280° C., or about 300° C.

In some embodiments, the method of converting hydrocarbons is carried out at a pressure within the range of about 1 bar to about 150 bars, e.g., about 5 bars to about 125 bars, or about 5 bars to about 100 bars, or about 5 bars to about 90 bars, or about 10 bars to about 80 bars, or about 15 bars to about 70 bars, or about 20 bars to about 60 bars, or about 25 bars to about 50 bars, or about 30 bars to about 45 bars, or about 35 bars to about 40 bars, or the pressure is about 15 bars, or about 20 bars, or about 25 bars, or about 30 bars, or about 35 bars, or about 40 bars, or about 45 bars, or about 50 bars, or about 55 bars, or about 60 bars, or about 65 bars, or about 70 bars.

### EXAMPLES

The Examples that follow are illustrative of specific embodiments of the disclosure, and various uses thereof. They are set forth for explanatory purposes only, and are not to be taken as limiting the disclosure.

### Example 1. SPA Catalyst Composition Preparation

111.5 g phosphoric acid (113% concentration) at 45° C. was added to a mixing bowl. 26.6 g of a dry particulate material comprising phosphoric acids and silicon phosphates (prepared by calcining a mixture of amorphous silica and phosphoric acid (113% concentration) present in a ratio within the range of about 1:2 to about 1:4, calculated on a weight basis), and 38.5 g diatomaceous earth were then added to the bowl and mixed in a high speed mechanical mixer for several minutes. The mixture was extruded using a hydraulic press, then calcined in air at a temperature for a time according to Table 1, providing SPA catalyst composition 1 (SPA-1). SPA-2 was prepared similarly, but the amount of diatomaceous earth was adjusted to provide a H₃PO₄:diatomaceous earth ratio of 3.60.

Comparative catalyst compositions C1 and C2 were prepared as described above for SPA-1 and SPA-2, but the dry particulate material was excluded.

TABLE 1
SPA Catalyst Compositions

| | | | | Calcina- | |
|---|---|---|---|---|---|
| | | Rework | Calcina- | tion Tem- | Acid |
| | | Component | tion Time | perature | Content |
| Catalyst | H₃PO₄:D.E.* | (wt. %)* | (min) | (° C.) | (wt. %) |
| SPA-C1 | 3.27 | 0 | 30 | 320 | 73-75 |
| SPA-1 | 3.27 | 15% | 30 | 320 | 73-75 |
| SPA-C2 | 3.60 | 0 | 30 | 320 | 75-78 |
| SPA-2 | 3.60 | 15% | 30 | 320 | 75-78 |

*Calculated as weight percentages of the formable mixture, before calcination

### Example 2. SPA-Catalyzed Olefin Oligomerization

SPA catalyst compositions prepared according to Example 1 were placed in a reactor. A feed containing 45 wt. % propane and 55 wt. % propylene, maintained at a moisture level of 510 ppm, was passed through the catalyst bed at a linear hourly space velocity (LHSV) of 2.8 h⁻¹. The temperature and the pressure of the catalyst bed were maintained at 216° C. and 65 bars. Table 2 shows the propylene conversion after 23, 47, 71, 95, 119, 143, 167, 191, and 215 hours on stream.

TABLE 2
Propylene Conversion with SPA Catalysts

| | |
|---|---|
| Time on | Propylene Conversion (%) |

| Stream (h) | SPA-C1 | SPA-1 | SPA-C2 | SPA-2 |
|---|---|---|---|---|
| 23 | 91.7 | 91.7 | 93.1 | 93.8 |
| 47 | 90.6 | 94.5 | 92.4 | 93.2 |
| 71 | 89.5 | 91.1 | 91.7 | 93.1 |
| 95 | 88.0 | 90.1 | 91.1 | 92.7 |
| 119 | 87.1 | 89.4 | 90.4 | 92.1 |
| 143 | 85.9 | 88.6 | 89.4 | 91.7 |
| 167 | 84.5 | 87.8 | 88.5 | 91.2 |
| 191 | 83.2 | 86.7 | 87.6 | 90.4 |
| 215 | 82.1 | 85.8 | 86.8 | 89.6 |

The data shown in Table 2 demonstrate that including a rework composition in the mixture and increasing the ratio of phosphoric acid to silica in the mixture improves both the average performance and deactivation rate of the catalyst composition.

### Example 3. SPA Catalyst Composition Crush Strength

A rework component comprising phosphoric acids and silicon phosphates, prepared by calcining a mixture of amorphous silica and phosphoric acid (113% concentration) present in a ratio within the range of about 1:2 to about 1:4, calculated on a weight basis, was crushed into particles using a hammer mill with a screen size ranging from 1.7 mm to 4.7 mm.

SPA catalyst compositions were prepared similarly to SPA-1 of Example 2, using hammer-milled rework components, as provided in Table 3. The crush strength and loss on attrition were determined for each calcined catalyst composition. Results are shown in Table 3.

TABLE 3

| Screen | Particles < | Particles < | Crush | Loss on |
|---|---|---|---|---|
| Size | 0.85 mm | 0.11 mm | Strength | Attrition |
| (mm) | (wt. %) | (wt. %) | (lbs/mm) | (%) |
| 1.7 mm | 86 | 26 | 1.7 ± 0.1 | 1.11 |
| 3.2 mm | 84 | 11 | 0.92 ± 0.03 | 1.12 |
| 4.7 mm | 80 | 5 | 1.1 ± 0.2 | 1.11 |

The data shown in Table 3 demonstrate that including a crushed rework component improves the physical characteristics of the catalyst composition, including compositions prepared from mixtures having a relatively high H₃PO₄:SiO₂ ratio, which would otherwise be disadvantageous.

What is claimed is:

1. A method for preparing a solid phosphoric acid catalyst composition, the method comprising
providing a formable mixture comprising
a phosphate source present in the formable mixture in an amount within the range of about 50 wt. % to about 85 wt. %, calculated as H₃PO₄;
a siliceous support material source present in the formable mixture in an amount within the range of about 8 wt. % to about 35 wt. %, calculated as SiO₂, for example, such that the ratio of the phosphate source to the siliceous support material source is within the range of about 2.9:1 to about 4.5:1, calculated on a weight basis as H₃PO₄:SiO₂; and
a dry particulate material present in the formable mixture in an amount within the range of about 2 wt % to about 25 wt %, the dry particulate material comprising silica;
one or more silicon phosphates; and/or
a mixture comprising one or more phosphoric acids, one or more silicon phosphates, and, optionally, a siliceous support material;
wherein the amount of silicon in the dry particulate material is at least about 15 wt. %, calculated as SiO₂ on a calcined basis;
forming the mixture; and
calcining the formed mixture.

2. A method according to claim 1, wherein the dry particulate material is a rework material.

3. A method according to claim 1, wherein the ratio of the phosphate source to the siliceous support material source is within the range of about 2.95:1 to about 4.5:1, calculated on a weight basis as H₃PO₄:SiO₂.

4. A method according to claim 1, wherein the ratio of the phosphate source to the siliceous support material source is within the range of 4.05:1 to 4.45:1, calculated on a weight basis as H₃Po₄:SiO₂.

5. A method according to claim 1, wherein the formed mixture is calcined at a temperature within the range of about 250° C. to about 420° C.

6. A method according to claim 1, wherein the formed mixture is calcined for a period of time within the range of about 20 minutes to about 4 hours.

7. A method according to claim 1, wherein the siliceous support material source comprises, diatomaceous earth, infusorial earth, ciliate earth, fuller's earth, kaolin, celite, artificial porous silica, or any mixture thereof.

8. A catalyst composition comprising,
one or more phosphoric acids
one or more silicon phosphates;
one or more additional inorganic phosphates; and
a siliceous support material,
wherein the amount of phosphate in the calcined solid phosphoric acid catalyst composition is within the range of about 60 wt % to about 80 wt % calculated as H₃PO₄ on a calcined basis.

9. The catalyst composition of claim 8, wherein the amount of phosphate in the calcined solid phosphoric acid catalyst composition is within the range of about 74.5 wt % to about 76.5 wt % calculated as H₃PO₄ on a calcined basis.

10. The catalyst composition of claim 8, further comprising at least 7 wt % of a pore forming material.

11. The catalyst composition claim 8, wherein the composition comprises an amount of silicon orthophosphate and, optionally, an amount of silicon phyrophosphate, wherein the integrated XRD reflectance intensity ratio of silicon orthophosphate to silicon pyrophosphate in the composition is at least about 5:1.

12. The catalyst composition of claim 8, wherein the composition has a total pore volume of at least about 0.17 cm³ per gram of the composition, wherein at least about 15 cm³ per gram is due to pores having a diameter of at least about 1 μm.

13. A method for converting hydrocarbons, the method comprising contacting a hydrocarbon feed with the catalyst composition, said catalyst composition comprising,
one or more phosphoric acids
one or more silicon phosphates;
one or more additional inorganic phosphates; and
a siliceous support material,
wherein the amount of phosphate in the calcined solid phosphoric acid catalyst composition is within the range of about 60 wt % to about 80 wt % calculated as H3PO4 on a calcined basis.

14. A method according to claim 13, wherein the hydrocarbon conversion is an olefin oligomerization or aromatic hydrocarbon alkylation.

15. A method according to claim 13, wherein the hydrocarbon feed comprises C3 hydrocarbon, C4 hydrocarbons, or any mixture thereof.
